(19) United States
(12) Patent Application Publication
(10) Pub. No.: US 2025/0090074 A1
(43) Pub. Date: Mar. 20, 2025

(54) IMPLANTABLE ECOG ELECTRODE, MANUFACTURING METHOD THEREOF, AND READABLE STORAGE MEDIUM

(71) Applicant: SHENZHEN WE-LINKING MEDICAL TECHNOLOGY CO., LTD., Shenzhen City (CN)

(72) Inventors: Chunshan DENG, Shenzhen City (CN); Tao SU, Shenzhen City (CN); Qingxia LI, Shenzhen City (CN); Bingjie ZHANG, Shenzhen City (CN); Qing GAO, Shenzhen City (CN); Liu YUAN, Shenzhen City (CN)

(21) Appl. No.: 18/961,903

(22) Filed: Nov. 27, 2024

Related U.S. Application Data
(63) Continuation of application No. PCT/CN2024/133037, filed on Nov. 19, 2024.

(30) Foreign Application Priority Data
Nov. 29, 2023 (CN) 202323249824.6
Dec. 21, 2023 (CN) 202311787313.1

(51) Int. Cl. A61B 5/293

(52) U.S. Cl. CPC A61B 5/293; A61B 2562/0209; A61B 2562/028; A61B 2562/046; A61B 2562/125; A61B 2562/164

(57) ABSTRACT

An implanted ECOG electrode, a manufacturing method thereof, and a computer-readable storage medium are provided in the application. The implanted ECOG electrode includes an electrode contact region and a welding pad region. The electrode contact region includes electrode contacts. A hollowed-out portion is disposed a position, except for an end portion of the conductive wire, between adjacent two electrode contacts, and the hollowed-out portion penetrates through a top surface and a bottom surface of the electrode contact region.

### CROSS-REFERENCE TO RELATED APPLICATION

This application is a Continuation of International Application No. PCT/CN2024/133037 filed on Nov. 19, 2024, which claims the benefit of priority of Chinese Patent Application No. 202323249824.6 filed with Chinese Patent Office on Nov. 19, 2023 and Chinese Patent Application No. 202311787313.1 filed with Chinese Patent Office on Dec. 21, 2023, the entire contents of which are incorporated herein by reference.

### TECHNICAL FIELD

The present application relates to the field of brain-machine interface technologies, and more particularly, to an implanted ECOG electrode, a manufacturing method thereof, and a readable storage medium.

### BACKGROUND

The brain-machine interface is an important technology for directly coupling the brain to a computer or other external devices through a sensing terminal, so that the extraction and the decoding of brain signals are finally converted into command signals that can be used to manipulate the external devices without relying on conventional output channels. Among them, one of the key factors of the brain-machine interface is information extraction, that is, information in the biological brain is read out. Currently, there are two kinds of information extraction methods, namely, non-implantable and implantable information extraction methods. In the non-implantable method, the electroencephalogram (referred briefly to as EEG) data is read by means of an EEG cap worn on the scalp. In the implantable method, the EEG data is obtained through the technologies including an implantable microelectrode array, a deep brain electrode, and a semi-implantable electrocorticography (referred briefly to as ECOG) electrode. Among these technologies, the ECOG electrodes are widely used in the field of brain-machine interfaces due to their high signal resolution, relatively long-term stability, and relatively little intrusion. ECoG measures sync signals of cortical neurons by implanting an array of electrodes in a subdural space of the brain. These signals are mainly composed of low-frequency components (less than 200 Hz (Hertz)), with amplitudes ranging from microvolts to millivolts. Compared with conventional hard multi-channel electrode array (referred briefly to as MEA) electrodes, the ECOG electrodes are made of flexible material and develop towards ultra-high density recording, large-range recording, miniaturization, and high biocompatibility.

### SUMMARY

The existing flexible electrode has the problems that the structure is unreasonable, the manufacturing process is complex, and the requirement for equipment is higher.

In view of the above, according to embodiments of the present application, it is provided an implantable ECoG electrode, a manufacturing method thereof, and a readable storage medium for improving at least one of the above technical problems.

In a first aspect, according to embodiments of the present application, it is provided an implanted semi-implantable electrocorticography (ECOG) electrode including an electrode contact region and a welding pad region; in which the electrode contact region is provided on a first side of the electrode, and the electrode contact region includes a plurality of electrode contacts; the welding pad region is disposed on a second side of the electrode remote from the first side, and the welding pad region includes a plurality of welding spots; each of the plurality of electrode contacts and a corresponding one of the plurality of welding spots are connected by a conductive wire; the electrode contact region includes a hollowed-out portion that is disposed a position, except for an end portion of the conductive wire, between adjacent two of the plurality of electrode contacts, and the hollowed-out portion penetrates through a top surface and a bottom surface of the electrode contact region.

In a second aspect, according to embodiments of the present application, it is provided a manufacturing method of a flexible electrode, the method including:
providing an electrode flexible support layer on a surface of a silicon wafer substrate;
disposing a photoresist on the electrode flexible support layer, in which the photoresist covers a part of a surface of the electrode flexible support layer;
forming a first metal layer on the electrode flexible support layer via deposition, in which the first metal layer includes a metal layer on the photoresist and an electrode structure metal layer on the electrode flexible support layer, and the electrode structure metal layer includes electrode contacts, welding spots, and a conductive wire between each of the electrode contacts and a corresponding one of welding spots;
removing the photoresist;
providing an encapsulation layer on the electrode flexible support layer, and removing portions of the encapsulation layer above the electrode contacts and the welding spots; and
peeling the electrode flexible support layer from the silicon wafer substrate to obtain the flexible electrode.

In a third aspect, according to embodiments of the present application, it is provided a computer-readable storage medium having programs stored thereon, when executed by a single-core processer or a multi-core processor, causes the single-core processor or the multi-core processor to perform the above-mentioned method.

### BENEFICIAL EFFECT

The implanted ECoG electrode provided in the embodiments of the present application has the following advantageous effects. Firstly, the electrode contact region of the implanted ECOG electrode provided in the embodiments of the present application includes hollowed-out portions, so that the flexibility of the electrode and/or the passage of cerebrospinal fluid may be improved. So, cerebrospinal fluid circulation is promoted, so that it may be beneficial to promoting the removal of metabolites, thereby reducing blockage, and helping to reduce microbial colonization. Further, the risk of infection is reduced, thus helping to maintain a healthy brain environment, significantly reducing the risk of brain diseases, and providing patients with extra health protection. In addition, this hollowed-out design also reduces the actual contact area between the electrode and the tissue, thereby further reducing the risk of scar formation in the brain, avoiding excessive nerve scars from hindering the electrode recording effect, and reducing the impact of scars on the quality of signals collected by the electrodes, which is conducive to long-term implant health. And, the hollowed-out portions may function as anchor points, thereby improving the stability of the electrode on the cortex. So, the electrode is difficult to shift and/or fall off, thereby improving the effectiveness of the electrode. Secondly, when the electrode contacts of the implanted ECOG electrode of the present application include small contacts and large contacts, the electrode may be integrated with recording and stimulation functions. That is, the signal acquisition may be performed while the signal stimulation is performed, and thus the electrode has convenience, high efficiency, good stability, and good reliability, which improves the resolution and accuracy of signal acquisition. Further, miniaturization and lightness may be realized, and flexibility and biocompatibility are also realized. In addition, the customized electrode array is allowed to be designed based on clinical needs Thirdly, The flexible substrate and the encapsulation layer of the implanted ECOG electrode of the present application are both polyimide layers, and are characterized by good biocompatibility, simple forming process, and good economy. Fourthly, the metal layer of the implanted ECoG electrode of the present application includes a gold layer, a tantalum layer, a niobium layer, an indium layer, a tungsten layer, a platinum layer, and/or a titanium layer. And, the metal layer has no leakage risk, is harmless to the human body, and thus has good safety. Fifthly, the implanted ECOG electrode of the present application is simple in structure, convenient to use, and wide in application range.

### BRIEF DESCRIPTION OF THE DRAWINGS

In order that the technical solution of the embodiments of the present application may be explained more clearly, a brief description will be given below of the accompanying drawings required for use in the embodiments. The accompanying drawings, which are incorporated in and constitute a part of the specification, illustrate embodiments consistent with the present application and, together with the description, serve to illustrate the technical solution of the present application. It is to be understood that the drawings illustrate only certain embodiments of the present application and are therefore not to be construed as limiting the scope of protection, and that other related drawings may be obtained from these drawings without involving any inventive effort to those skilled in the art. Also throughout the drawings, like reference numerals refer to like parts. In the drawings:

FIG. 1 shows a schematic structural diagram of an implanted ECoG electrode according to embodiments of the present application.

FIG. 2 shows an enlarged structural diagram at detail A according to embodiments of the present application.

FIG. 3 shows another schematic structural diagram of the implanted ECoG electrode according to embodiments of the present application.

FIG. 4 shows an enlarged structural diagram at detail B according to embodiments of the present application.

FIG. 5 shows yet another schematic structural diagram of the implanted ECoG electrode according to embodiments of the present application.

FIG. 6 shows an enlarged structural diagram at detail C according to embodiments of the present application.

FIG. 7 shows yet another schematic structural diagram of the implanted ECoG electrode according to embodiments of the present application.

FIG. 8 shows an enlarged structural diagram at detail D according to embodiments of the present application.

FIG. 9 shows a sectional schematic view in a lengthwise direction of an electrode contact region of an ECOG electrode according to embodiments of the present application.

FIG. 10 is a schematic structural diagram of a manufacturing device for a flexible electrode according to embodiments of the present application.

FIG. 11 is a schematic flow diagram of a manufacturing method of a flexible electrode according to embodiments of the present application.

FIG. 12 is a sectional view of a flexible electrode during manufacture according to embodiments of the present application.

FIG. 13 is another sectional view of the flexible electrode during manufacture according to embodiments of the present application.

FIG. 14 is a sectional view of a flexible electrode during manufacture according to embodiments of the present application.

FIG. 15 is another sectional view of the flexible electrode during manufacture according to embodiments of the present application.

FIG. 16 is a sectional view of a flexible electrode during manufacture according to embodiments of the present application.

FIG. 17 is another sectional view of the flexible electrode during manufacture according to embodiments of the present application.

FIG. 18 is a sectional view of a flexible electrode during manufacture according to embodiments of the present application.

FIG. 19 is a method flow diagram of removing portions of an encapsulation layer which are above electrode contacts and welding spots in accordance with embodiments of the present application.

FIG. 20 is a sectional view of a flexible electrode during manufacture according to embodiments of the present application.

FIG. 21 is another sectional view of the flexible electrode during manufacture according to embodiments of the present application.

FIG. 22 is a sectional view of a flexible electrode during manufacture according to embodiments of the present application.

FIG. 23 is another sectional view of the flexible electrode during manufacture according to embodiments of the present application.

FIG. 24 is a sectional view of a flexible electrode during manufacture according to embodiments of the present application.

FIG. 25 is another sectional view of the flexible electrode during manufacture according to embodiments of the present application.

FIG. 26 is a sectional view of a flexible electrode according to embodiments of the present application.

FIG. 27 is another sectional view of the flexible electrode according to embodiments of the present application.

FIG. 28 is a structural diagram of a flexible electrode according to embodiments of the present application.

### DETAILED DESCRIPTION

Exemplary embodiments of the present application will be described in more detail below with reference to the accompanying drawings. While exemplary embodiments of the present application are shown in the drawings, it is to be understood that the application may be embodied in various forms and should not be limited to the embodiments set forth herein. Rather, these embodiments are provided to enable a more thorough understanding of the present application and to convey the scope of the application to those skilled in the art in its entirety.

In the description of embodiments of the present application, the terms such as “including” or “having,” or the like, are intended to indicate the presence of the disclosed features, integers, steps, acts, components, parts, or combinations thereof, but do not preclude the presence or addition of one or more other features, integers, steps, acts, components, parts, or combinations thereof in the present specification.

Unless otherwise specified, “/” means “or”. For example, A/B may mean A or B. “And/or” in this specification describes only an association relationship for describing associated objects and represents that three relationships may be included. For example, A and/or B may represent the following three cases: only A is included, both A and B are included, and only B is included.

The terms “first” and “second” are only used for descriptive purposes to distinguish the same or similar technical features, and cannot be understood as indicating or implying relative importance or implicitly indicating the number of indicated technical features. Thus, the features defined as “first” and “second” can explicitly or implicitly include one or more of the described features. In the description of the present application, “multiple” means two or more, unless otherwise specifically defined.

In the descriptions of the embodiments of this application, unless otherwise specified, “a plurality of” means two or more.

Directional terms such as “first side”, “second side”, “length”, “width”, “middle region”, etc. mentioned in this application are only used as a reference to the accompanying drawings. Therefore, the directional terms used are used to illustrate and understand this application, but not to limit this application.

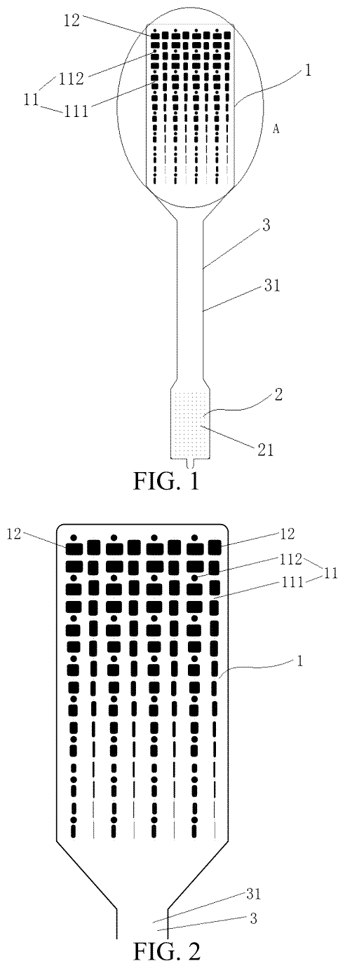

As shown in FIGS. 1 to 4, the implanted ECOG electrode of the present application includes an electrode contact region 1 and a welding pad region 2.

The electrode contact region 1 is provided on a first side of the electrode, and the electrode contact region 1 includes a plurality of electrode contacts 11.

The welding pad region 2, which includes a plurality of welding spots 21, is provided on a second side of the electrode remote from the first side.

Each electrode contact 11 and a corresponding welding spot 21 are connected by a conductive wire 3.

The electrode contact region 1 includes a hollowed-out portion 12. The hollowed-out portion 12 is disposed a position, except for an end portion of the conductive line 3, between the two adjacent electrode contacts 11. The hollowed-out portion extends through a top surface and a bottom surface of the electrode contact region 1

In use, the electrode contact region 1 may be in contact with the subdural cortex of the brain. The welding pad region 2 may be soldered to a connector of a Flexible Circuit Board (referred briefly to as FPC) and is responsible for leading out the EEG signals collected by the contacts. The hollowed-out portions 12 may improve the flexibility of the electrode and/or the passage of cerebrospinal fluid. And, the hollowed-out portions 12 may function as anchor points, thereby improving the stability of the electrode on the cortex. Therefore, the electrode is difficult to shift and/or fall off, thereby improving the effectiveness of the electrode.

In embodiments, as shown in FIGS. 1 to 4, the number of the hollowed-out portions 12 is multiple, and the multiple hollowed-out portions 12 are arranged at intervals, so that the flexibility of the electrodes and/or the passage of cerebrospinal fluid may be further improved. In this way, the stability of the electrodes on the cortex may be further improved, so that the electrodes are not easily shifted and/or fallen off, thereby further improving the effectiveness of the electrodes.

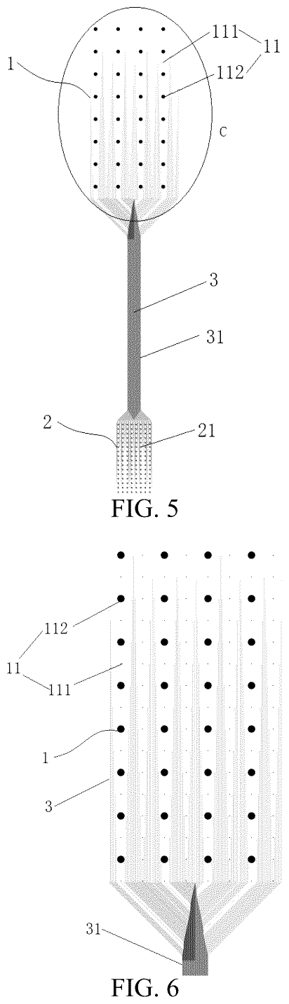

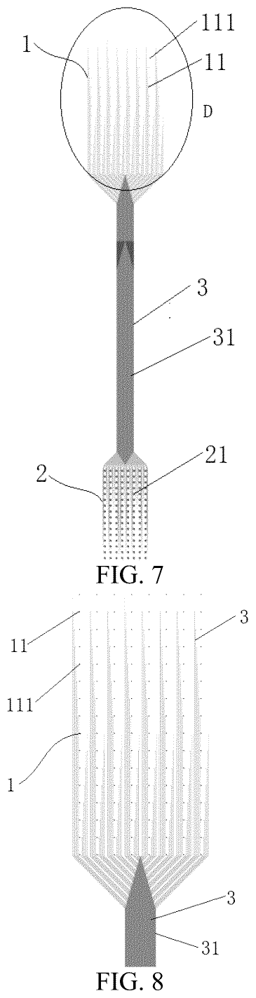

In a particular embodiment, as shown in FIGS. 1-8, the electrode contact 11 includes a small contact 111 and/or a large contact 112. Therein, the small contact may be used for recording, for example, signal acquisition. The large contact may be used for stimulation, for example, signal stimulation. When the electrode contacts include the small contacts and the large contacts, the electrode may be integrated with both the recording function and the stimulation function. That is, the signal acquisition may be performed while the signal stimulation is performed, and thus the electrode has convenience, high efficiency, good stability, and good reliability.

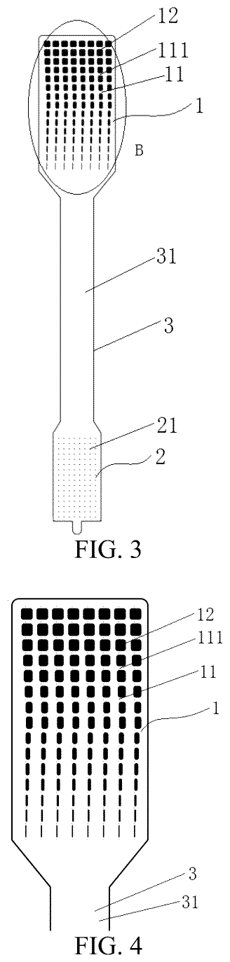

In embodiments, as shown in FIGS. 3, 4, 7, and 8, when the plurality of electrode contacts 11 are the plurality of small contacts 111, the plurality of small contacts 111 are arranged in an array, and the every adjacent two small contacts 111 are arranged at equal intervals. So, the recording effect of the electrodes may be improved. For example, the uniformity of the signal acquisition of the electrodes is good and the sensitivity of the electrodes is high.

In embodiments, when the plurality of electrode contacts 11 are the plurality of large contacts 112, the plurality of large contacts 112 are arranged in an array, and the every adjacent two large contacts 112 are arranged at equal intervals. So, the stimulation effect of the electrodes may be improved. For example, the uniformity of the signal stimulation of the electrodes is good, and the sensitivity thereof is high.

In embodiments, as shown in FIGS. 1, 2, 5, and 6, when the plurality of electrode contacts 11 are the plurality of small contacts 111 and the plurality of large contacts 112, the plurality of small contacts 111 are arranged in an array and the plurality of large contacts 112 are arranged in an array. The array of small contacts 111 and the array of large contacts 112 are disposed in a staggered arrangement, and every two adjacent small contacts 111 are arranged at equal intervals and/or every two adjacent large contacts 112 are arranged at equal intervals, which can improve the recording and stimulating effects of the electrodes. For example, the signal acquisition uniformity of the electrodes is good, the signal stimulation uniformity thereof is good, and both the signal acquisition and the signal stimulation have high sensitivity.

In embodiments, as shown in FIGS. 1 to 8, an outer diameter of the small contact 111 ranges from 5 micron (i.e., μm) to 500 μm, and the recording effect of the electrode is good. An outer diameter of the large contact 112 ranges from 500 μm to 2500 μm, and the stimulation effect of the electrode is good.

In embodiments, as shown in FIGS. 1, 3, 5, and 7, the number of the electrode contacts 11 is the same as the number of the welding spots 21, so that the connection using the conductive wires 3 may be facilitated.

In embodiments, as shown in FIGS. 1 to 8, the number of electrode contacts ranges from 16 to 1024, and the recording effect and/or the stimulation effect of the electrodes is good. For example, the number of electrode contacts is 32, 64, 128, or 256.

In embodiments, as shown in FIGS. 1 to 8, a width of a middle region 31 of the conductive line 3 is smaller than a width of the electrode contact region 1 and a width of the welding pad region 2, and the width of the welding pad region 2 is smaller than the width of the electrode contact region 1. So, the compact layout of the electrodes may be improved, thereby facilitating the miniaturization of the electrodes.

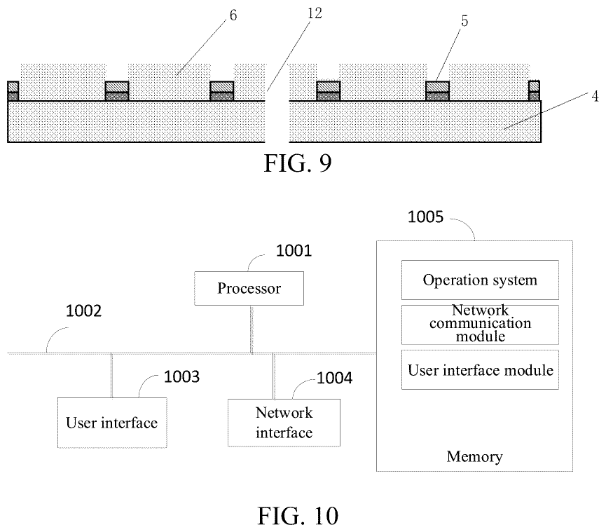

In embodiments, as shown in FIG. 9, the electrodes are provided to be a sheet-like film. The electrode includes a flexible substrate 4, a metal layer 5 and an encapsulation layer 6.

A flexible substrate 4 is provided at a bottom of the electrode;
the encapsulation layer 6 is provided on a top of the electrode.

The metal layer 5 includes electrode contacts 11 and welding spot 21 and the conductive wire 3. The conductive wire 3 is provided between the flexible substrate 4 and the encapsulation layer 6. The electrode contacts 11 and the welding spots 21 are exposed from the encapsulation layer 6.

The hollowed-out portion 12 extends through a top surface of the encapsulation layer 6 and a bottom surface of the flexible substrate 4 in the electrode contact region 1.

The flexibility of the electrodes may be improved by using the flexible substrate 4. The safety and the strength of the electrodes may be improved by the encapsulation layer 6. The electrode contacts 11 are exposed out of the encapsulation layer 6 to be able to contact the tissue and to perform recording and/or stimulation of electrical signals. The welding spots 21 are exposed out of the encapsulation layer 6 to be brought into contact with corresponding welding spots on the flexible circuit board.

In embodiments, as shown in FIG. 9, both the flexible substrate 4 and the encapsulation layer 6 are polyimide layers, which are characterized by good biocompatibility, simple forming process and good economy.

In embodiments, as shown in FIG. 9, the metal layer 5 includes a gold layer, a tantalum layer, a niobium layer, an indium layer, a tungsten layer, a platinum layer, and/or a titanium layer, so there is no leakage risk. In this way, the metal layer 5 is not harmful to the human body, and thus has good safety.

In embodiments, as shown in FIG. 9, a thickness of the encapsulation layer 6 is less than or equal to a thickness of the flexible substrate 4, so that the supporting stability and the reliability of the electrodes may be improved.

In embodiments, as shown in FIG. 9, the thickness of the flexible substrate 4 ranges from 0.1 μm to 100 μm. So, the electrode has good support stability and good reliability. The thickness of the encapsulation layer 6 ranges from 0.1 μm to 100 μm. So, the electrode has good encapsulation stability and good reliability.

In embodiments, as shown in FIG. 9, the thickness of the metal layer 5 ranges from 10 nm to 1000 nm, and thus the metal performance is good in stability and also is good in reliability.

In the present application, in usage, the electrode contact region 1 is brought into contact with the subdural cortex of the brain, and the welding pad region 2 is welded to the connector of the flexible circuit board. The hollowed-out portions 12 may improve the flexibility of the electrodes and/or the passage of cerebrospinal fluid. And, the hollowed-out portions 12 may function as anchor points, thereby improving the stability of the electrodes on the cortex, making the electrodes difficult to shift and/or fall off, and further improving the effectiveness of the electrodes. At the same time, the signal acquisition and/or stimulation is performed according to the actual needs of the electrodes.

As shown in FIG. 10, FIG. 10 is a schematic structural diagram of a manufacturing device for a flexible electrode according to an embodiment of the present application.

It should be noted that FIG. 10 is a schematic structural diagram of a hardware operating environment for the manufacturing device of the flexible electrode. The basic manufacturing device for the flexible electrode in the embodiments of the present application may be a terminal device such as a personal computer (referred briefly to as PC) or a portable computer.

As shown in FIG. 10, the manufacturing device for the flexible electrode may include a processor 1001, such as a CPU, a network interface 1004, a user interface 1003, a memory 1005, and a communication bus 1002. The communication bus 1002 is used to implement connection communication among these components. The user interface 1003 may include a Display, and an input unit such as a Keyboard. The user interface 1003 may also include a standard wired interface, and a wireless interface. The network interface 1004 may optionally include a standard wired interface, a wireless interface (e.g., a WI-FI interface). The memory 1005 may be a high-speed Random Access Memory (referred briefly to as RAM) memory or may be a non-volatile memory, such as a disk memory. The memory 1005 may optionally also be a memory device independent of the afore mentioned processor 1001.

It will be appreciated by those skilled in the art that the structure of the manufacturing device of the flexible electrode shown in FIG. 10 does not constitute a definition of the manufacturing device for the flexible electrode, and the manufacturing device for the flexible electrode may include more or less components than the illustrated components, or may combine certain components, or may have different component arrangements.

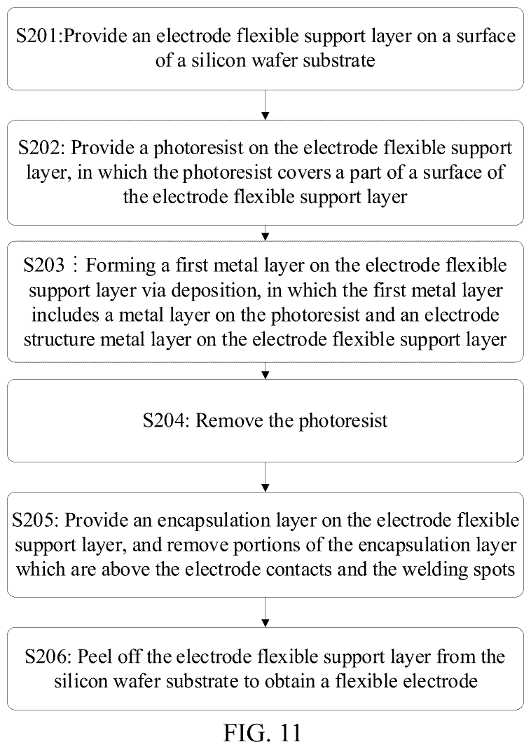

Referring to FIG. 11, FIG. 11 shows a method flow chart for manufacturing a flexible electrode according to an embodiment of the present application. In this flow, from a device viewpoint, the execution body may be one or more electronic devices. From a program viewpoint, the execution body may correspondingly be programs mounted on the electronic devices.

As shown in FIG. 11, an embodiment of the present application provides a manufacturing method of a flexible electrode, including steps S201 to S206.

At step S201, an electrode flexible support layer is provided on a surface of a silicon wafer substrate.

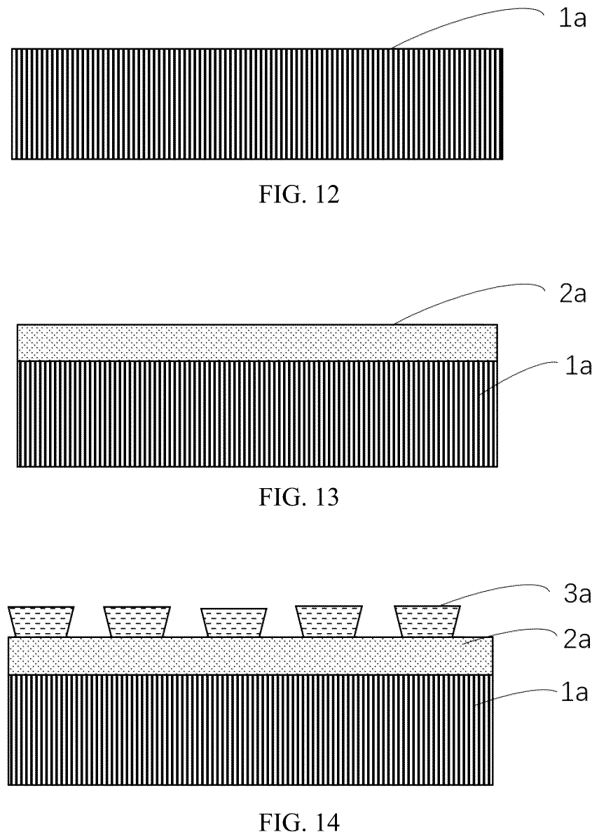

As shown in FIGS. 12 and 13, an electrode flexible support layer 2a may be provided on the surface of the silicon wafer substrate 1a. In embodiments of the present application, a thickness of the electrode flexible support layer may be between 1 μm and 1000 μm. The material of the electrode flexible support layer includes at least one of polyimide, SU-8, liquid crystal polymer, and Parylene-C. As an example, the thickness of the electrode flexible support layer 2a may be any value in the range of 1 μm, 2 μm, 4 μm, 6 μm and 9 μm, etc., or from 0.1 μm to 1000 μm. In a practical application, the electrode flexible support layer 2a may be coated on the silicon wafer substrate 1a. The coating method includes spin coating, spraying, and the like.

At step S202, a photoresist is provided on the electrode flexible support layer, and the photoresist covers a part of a surface of the electrode flexible support layer.

As shown in FIG. 14, the photoresist 3a may be provided on the electrode flexible support layer 2a in the present application. In the embodiments of the present application, the photoresist 3a with a predetermined pattern may be formed after the photoresist 3a is photoetched and developed on the electrode flexible support layer 2a. The regions that are uncovered by the photoresist 3a corresponds to the regions where the electrode structure metal layer of the obtained flexible electrode finished product is located. As a possible embodiment, the photoresist in the embodiments of the present application may be formed by a positive resist inversion process and an undercut process, and the section of the edge profile of the developed photoresist with the preset pattern is inverted trapezoidal. It should be noted that the section of the formed edge profile with the pattern after development is inverted trapezoidal, compared with a process in which the patterned edge profile has a square section, this shape allows a gap near the bottom layer after depositing and forming the metal layer, thereby facilitating liquid inflow, and facilitating subsequent stripping processes.

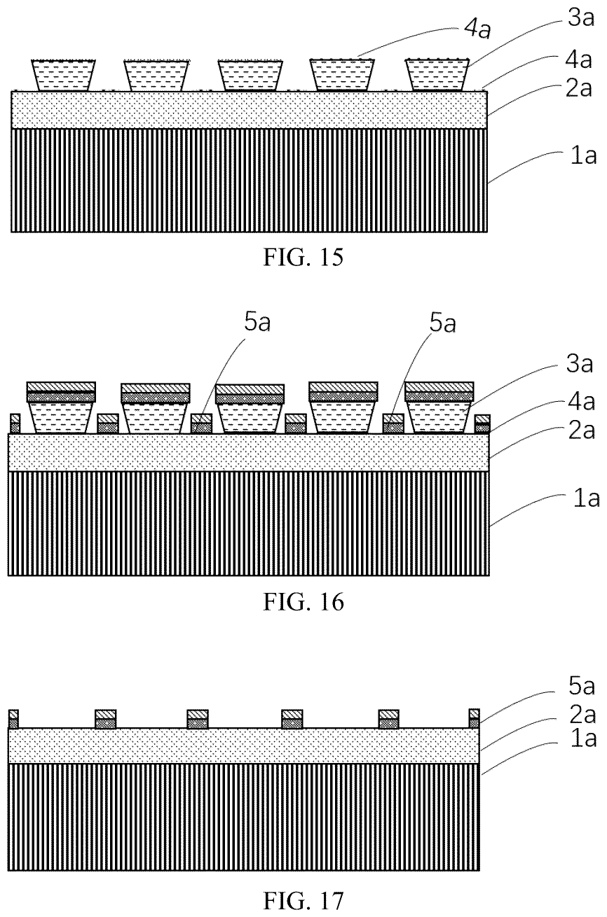

After the photoresist is provided on the electrode flexible support layer, as shown in FIG. 15, an upper surface of the electrode flexible support layer 2a covered with the photoresist 3a may be subjected to ionization treatment. As a possible embodiment, in the embodiments of the present application, it may perform a hydrogen plasma treatment on the upper surface of the electrode flexible support layer 2a covered with the photoresist 3a, so as to form structures 4a. The structures 4a are present on both the surface of the electrode flexible support layer and the surface of the photoresist. The structures 4a serve to increase the roughness of the upper surface of the electrode flexible support layer while surface impurities may be removed, thereby increasing the bonding force between the subsequently deposited metal and the upper surface of the electrode flexible support layer.

At step S203, a first metal layer is formed on the electrode flexible support layer via deposition, in which the first metal layer includes a metal layer on the photoresist and an electrode structure metal layer on the electrode flexible support layer.

As shown in FIG. 16, the first metal layer 5a may be formed on the electrode flexible support layer 2a after deposition in the present application. The first metal layer 5a includes the metal layer on the photoresist and the electrode structure metal layer on the electrode flexible support layer. After the formation of the first metal layer 5a, the structures 4a are no longer visible. In practical applications, in the present application, the first metal layer 5a is formed on the electrode flexible support layer 2a after deposition by a thin film deposition process.

It should be noted that the thin film deposition process may include any one of processes such as electron beam evaporation, thermal evaporation, and magnetron sputtering. In an embodiments of the present application, the electrode structure metal layer includes electrode contacts, welding spots, and a conductive wire between the electrode contact and the welding spot. A diameter of the electrode contact may be between 10 μm and 1500 μm. The first metal layer may include at least one of gold, aluminum, tungsten, platinum, and titanium. The thickness of the first metal layer may be between 1 nm and 2000 nm. In the present application, the sizes of the electrode contacts are designed at the micron level, which is closer to neuronal tissue. The sizes of the electrode contacts are closer to the cortical functional pillars of the study subject. The cortical functional pillar is between 100 μm and 500 μm in size, and is considered to be the basic unit of information processing. The sizes of the electrode contacts in the present application enable to obtain higher spatial resolution and finer information, and thus the electrode contacts have better biocompatibility.

At step S204, the photoresist is removed.

In practical applications, in the present application, it may use acetone or N-methylpyrrolidone (NMP) to strip the photoresist by heating the water bath, and the metal layer on the photoresist is stripped along with the photoresist. As an example, the product may be subjected to a heated water bath at 90° C. to strip the photoresist by N-methylpyrrolidone. As shown in FIG. 17, in the present application, after stripping the photoresist 3a by the acetone or N-methylpyrrolidone through the heated water bath, the electrode flexible support layer 2a remains the electrode structure metal layer that the first metal layer 5a is disposed on the electrode flexible support layer 2a.

At step S205, an encapsulation layer is provided on the electrode flexible support layer, and portions of the encapsulation layer which are above the electrode contacts and the welding spots are removed.

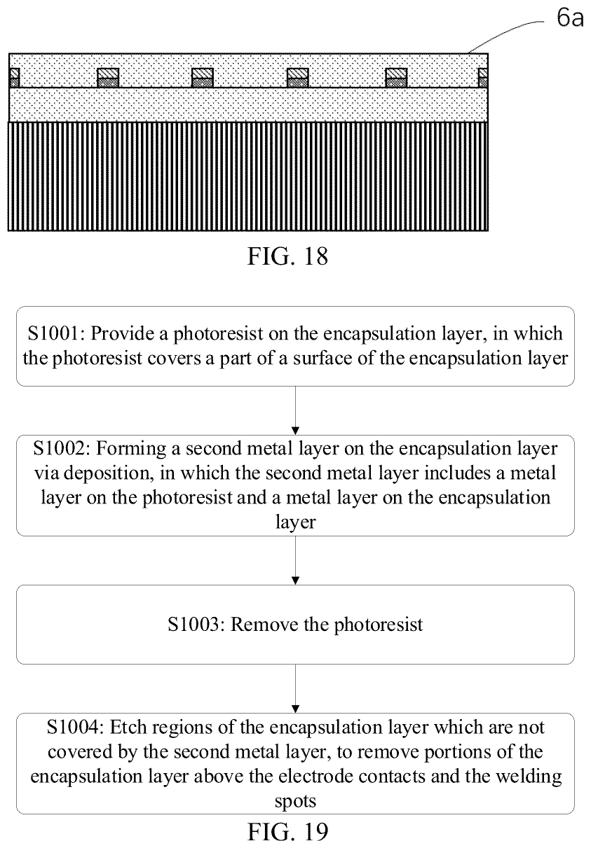

As shown in FIG. 18, in embodiments of the present application, it may further provide an encapsulation layer 6a on the electrode flexible support layer 2a, and the electrode structure metal layer in the first metal layer 5a is encapsulated in the encapsulation layer 6a. The material of the encapsulation layer 6a may include at least one of polyimide, SU-8, silicon carbide, liquid crystal polymer, Parylene-C, ceramic, and silicon dioxide.

In the present application, the encapsulation layer is provided on the electrode flexible support layer, and the portions of the encapsulation layer above the electrode contacts and the welding spots need to be removed. As a possible embodiment, the method of removing portions of the encapsulation layer above the electrode contacts and the welding spots provided by the embodiments of the present application may include steps S1001 to S1004.

At step S1001, a photoresist is provided on the encapsulation layer, and the photoresist covers a part of a surface of the encapsulation layer.

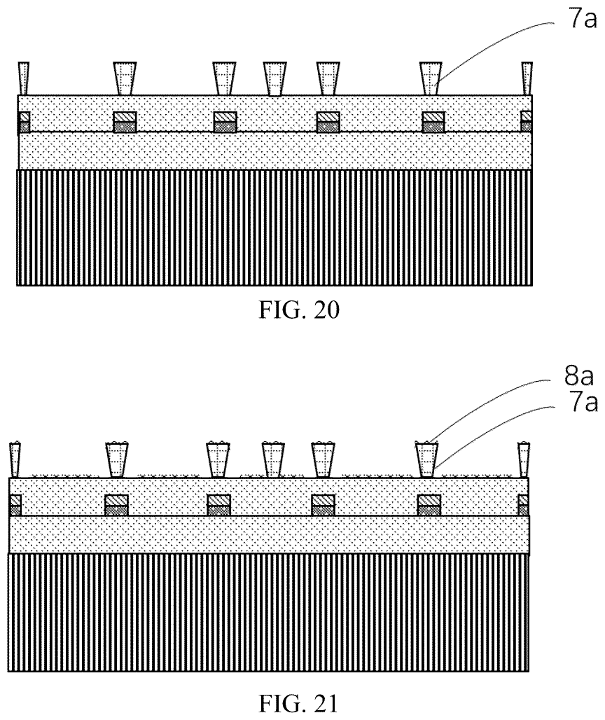

As shown in FIG. 20, the photoresist 7a may be provided on the encapsulation layer 6a, and the photoresist 7a covers a part of the surface of the encapsulation layer 6a. The photoresist 7a in the present embodiment may be formed by a positive resist inversion process and an undercut process, and a section of an edge profile of the developed photoresist with the preset pattern is inverted trapezoidal. It should be noted that the patterned edge profile formed after development has an inverted trapezoidal cross-section, compared with a process in which the patterned edge profile has a square section, this shape allows a gap near the bottom layer after depositing and forming the metal layer, thereby facilitating liquid inflow, and further facilitating subsequent stripping processes.

After the photoresist is provided on the electrode flexible support layer, as shown in FIG. 21, an upper surface of the encapsulation layer 6a covered with the photoresist 7a may be subjected to ionizing treatment. As a possible embodiment, in the embodiment of the present application, it may perform a hydrogen plasma treatment on the upper surface of the encapsulation layer 6a covered with the photoresist 7a to form the structures 8a. The structures 8a are present on both the surface of the encapsulation layer 6a and the surface of the photoresist 7a. The structures 8a serve to enhance the roughness of the upper surface of the encapsulation layer 6a while surface impurities may be removed, thereby increasing the bonding force between the subsequently deposited metal and the upper surface of the encapsulation layer 6a.

At step S1002, a second metal layer is formed on the encapsulation layer via deposition, in which the second metal layer includes a metal layer on the photoresist and a metal layer on the encapsulation layer.

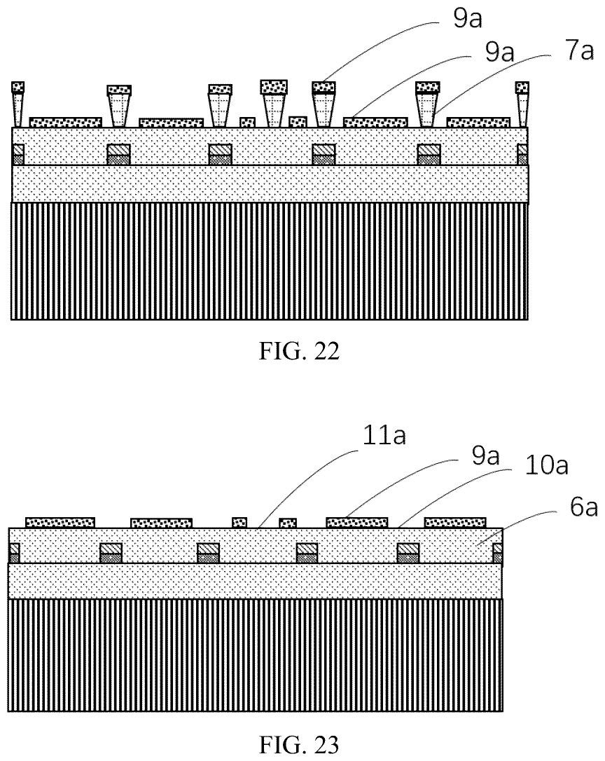

As shown in FIG. 22, in embodiments of the present application, it may deposit a second metal layer 9a on the encapsulation layer 6a. The second metal layer 9a includes a metal layer on the photoresist 7a and a metal layer on the encapsulation layer 6a. After the formation of the second metal layer 9a, the structures 8a are no longer visible. In the present application, it may deposit the second metal layer 9a on the encapsulation layer 6a by a physical vapor deposition process. The material of the second metal layer 9a may include at least one of cadmium, aluminum, copper, tungsten, platinum, and titanium.

At step S1003, the photoresist is removed.

As shown in FIG. 23, after the photoresist is removed in the present application, the metal layer on the photoresist is also removed along with the photoresist. The region formed by removing the second metal layer 9a and the photoresist 7a includes an electrode slotted region 11a and an region 10a corresponding to the electrode structure metal layer. The step S1003 of removing the photoresist is similar to the step S204 of removing the photoresist in the present application. For the implement of step S1003, the reference may be made to step S204.

At step S1004, regions of the encapsulation layer which are not covered by the second metal layer are etched, to remove portions of the encapsulation layer above the electrode contacts and the welding spots.

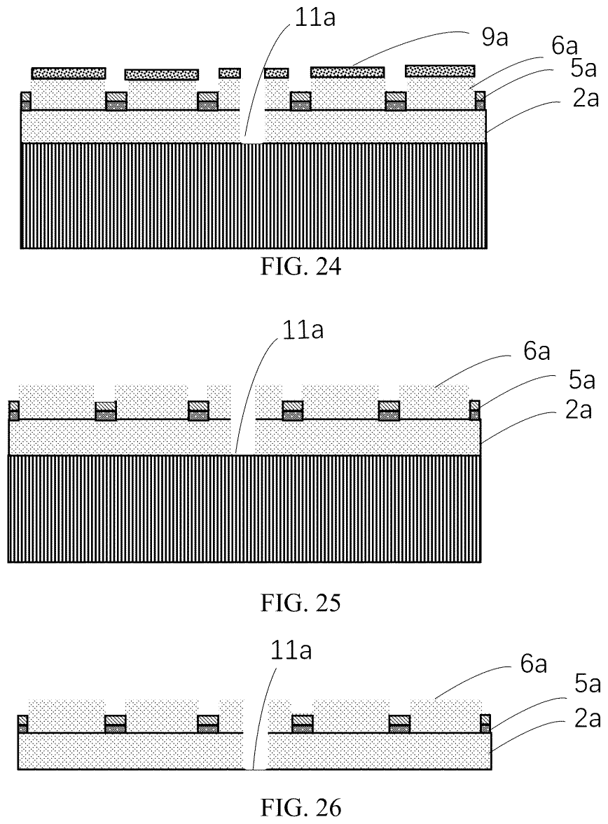

In the embodiments of the present application, it may use a reactive ion etching process to etch regions of the encapsulation layer that are not covered by the second metal layer. The regions of the encapsulation layer which are not covered by the second metal layer include an region 10a corresponding to the electrode structure metal layer and an electrode slotted region 11a. In the present application, the regions of the encapsulation layer which are not covered by the second metal layer are etched, so that the portions of the encapsulation layer above the electrode contacts and the welding spots may be removed, so as to obtain the product as shown in FIG. 24. The region 10a of the encapsulation layer 6a above the electrode contacts and the welding spots and the electrode flexible support layer 2a and the encapsulation layer 6a corresponding to the electrode slotted region 11a may be removed. Gases that may be used in the reactive ion etching process include oxygen; oxygen and sulfur hexafluoride; oxygen and carbon tetrafluoride, and the like, and examples of the present application are not limited herein.

According to the technical solution of the present application, the electrode slotted region 11a is arranged on the flexible electrode to better maintain a good environment of the brain. The opening design of the electrode slotted region 11a may promote cerebrospinal fluid flow, and help to maintain the health of the brain, and cleanness and normal function of the brain. In this way, the abnormal deposition and aggregation of harmful proteins is reduced, thereby reducing the risk of other brain diseases. In the technical solution of the present application, after etching the regions of the encapsulation layer which are not covered by the second metal layer, the second metal layer 9a may be removed to obtain the product shown in FIG. 25.

At step S206, the electrode flexible support layer is peeled off from the silicon wafer substrate to obtain a flexible electrode.

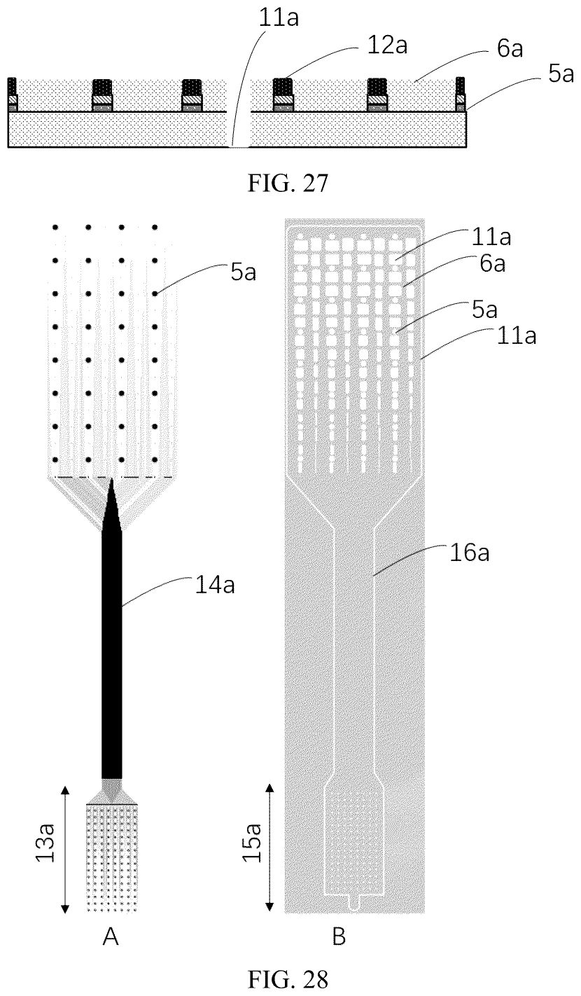

In the present embodiment, as shown in FIG. 26, the electrode flexible support layer 2a may be peeled off from the silicon wafer substrate 1a to obtain the flexible electrode. As a possible embodiment, as shown in FIG. 27, it is also possible to deposit metal on the flexible electrode by electroplating, so that a metal layer 12a is added to the electrode contacts 5a, so that the electrode contacts are flush with the encapsulation layer or the electrode contacts are higher than the encapsulation layer. In practical applications, in the present application, it may connect the flexible electrode to the PCB board through a connector and then to the electrochemical workstation through the PCB board. A suitable as solution, such Poly(3,4-ethylenedioxythiophene) (PEDOT) solution, or Poly(3,4-ethylenedioxythiophene) polystyrene sulfonate (PEDOT: PSS) solution, is prepared to deposit the metal on the formed flexible electrode by electroplating by means of electrochemical polymerization to form the metal layer 12a. It should be noted that the electrode contact region of the flexible electrode in the present application may be parallel to or higher than the insulating layer, which is more favorable for the acquisition of neural signals.

The flexible electrode made in the embodiments of the present application may be used to measure the electroencephalogram signal of the organism. The flexible electrode may be released in pure water after the flexible electrode is made.

In the embodiments of the present application, the structure of the obtained flexible electrode may be shown in FIG. 28. As shown in FIG. 28, the metal region of the flexible electrode includes electrode contacts 5a, welding spots connected to a Flexible Printed Circuit Board (FPC), and a lead wire region. The flexible electrode also includes an encapsulation layer 6, a hollowed-out electrode slotted region 11a, a welding pad region 13a, a single electrode profile slotted region 16a, an electrode contact-welding spot connection trace region 14a, and a welding-spot-region window pattern 15a. Here, the welding pad region 13a is used for soldering with the connector of the FPC. The sectional structure of the single electrode profile slotted region 16a coincides with that of the electrode slotted region 11.

The shape of the electrode contact in the embodiments of the present application may be one of a square shape, a rectangle shape, a triangle shape, a diamond shape, an elliptical shape, or a polygonal shape. The flexible electrode of the present application has at least one electrode contact, and the maximum number of the electrode contacts is 100,000. At least one electrode contact of the flexible electrode is used for signal acquisition. At least one electrode contact of the flexible electrode is used for electrical stimulation. The at least one electrode contact for electrical stimulation has a charge injection capacity (referred briefly to as CIC) of 10 mC/cm²˜−0.01 mC/cm². The at least one electrode contact for electrical stimulation has the CIC of 10 mC/cm²−100 mC/cm². The impedance of the electrode contact for stimulation may be between 100 ohms and 10 megohms. The shape of the opening in the flexible electrode may be one of square, rectangular, circular, elliptical or polygonal shapes.

The flexible electrode made in the embodiment of the present application is made of a flexible material, and an opening is provided in the electrode to reduce the strength of the electrode, so that the electrode is easily deformed, the flexibility of the electrode is increased, the adaptability and the flexibility of the electrode and the cortex tissue are improved, the risk of a potential inflammatory reaction is reduced, and the flexible electrode is more suitable for in-vivo recording for a long time.

In summary, according to the manufacturing method of a flexible electrode provided in the embodiments of the present application, a photoresist is provided on an electrode flexible support layer, and then a first metal layer is formed on the electrode flexible support layer via deposition. The first metal layer includes a metal layer on the photoresist and an electrode structure metal layer on the electrode flexible support layer. The photoresist is removed. The metal layer on the photoresist is also removed along with the photoresist. So, the step of the de-alloying treatment in the related art may be omitted, so that the manufacturing process of the flexible electrode is more simple and the requirement for the equipment is lower.

In the description of this specification, the description with reference to terms such as “some possible embodiment”, “some embodiments”, “examples”, “specific examples”, or “some examples”, and the like indicates that the specific feature, structure, material or characteristic described in conjunction with the embodiment or example is included in at least one embodiment or example of the present application. In this specification, the schematic representations of the aforesaid terms do not necessarily for the same embodiment or example. Moreover, the specific features, structures, materials or characteristics as described may be combined in any one or more embodiments or examples in a suitable manner. In addition, those skilled in the art may combine and group the different embodiments or examples and the features of the different embodiments or examples described in this specification without contradicting each other.

With respect to the flowchart of the method according to embodiments of the present application, certain operations are depicted as different steps performed in a certain sequence, and such a flowchart is illustrative rather than restrictive. Some of the steps described herein may be grouped together and performed in a single operation, some steps may be split into a plurality of sub-steps, and some steps may be performed in an order different from that shown herein. All the steps shown in the flowchart may be implemented in any manner by any circuit structure and/or tangible mechanism (e.g., by software running on a computer device, hardware such as logic functions implemented by a processor, chip and the like, and/or any combination thereof).

It will be understood by those skilled in the art that, in the method according to an embodiment, the sequence of the steps as presented does not imply a strict executing sequence and thereby does not constitute a restriction to the implementing process, and the specific executing sequence of the steps shall be determined by their function and possible internal logic.

According to the manufacturing method of the flexible electrode provided in the above embodiments, the embodiment of the present application further provides a flexible electrode. The flexible electrode is manufactured by the manufacturing method of the flexible electrode provided in the above embodiments.

It should be noted that the flexible electrodes in the embodiments of the present application are made by the processes of the embodiments of the foregoing methods and may achieve same effects and same functions. So, the details are not described herein.

According to some embodiments of the present application, it is provided a non-volatile computer storage medium for a manufacturing method of a flexible electrode, having machine-executable instructions stored thereon that, when executed by processor(s), cause the processor(s) to perform the method described in the above-described embodiments.

The computer-readable medium may be permanent and non-permanent, or removable and non-removable media, which can achieve the information storage by any method or technology. The information may be computer-readable instructions, data structures, program modules, or other data. Examples of the computer storage medium include, but are not limited to, a phase change memory (PRAM), a static random access memory (SRAM), a dynamic random access memory (DRAM), other types of random access memory (RAM), read-only memory (ROM), an electrically erasable programmable read-only memory (EEPROM), a flash memory or other memory technologies, a CD-ROM, a digital versatile disc (DVD) or other optical storage, and a magnetic cassette tape. The magnetic tape storage or other magnetic storage devices or any other non-transmission medium may be used to store information that can be accessed by computing devices. Furthermore, although the operations of the method of the present application are described in a specific order in drawings, it does not require or imply that the operations must be performed in the specific order, or that the desired result can only be achieved if all the operations as shown are performed. Additionally or alternatively, some steps may be omitted, multiple steps may be combined into one step for execution, and/or one step may be decomposed into multiple steps for execution.

It should also be noted that the embodiments in the present application and the features in the embodiments may be combined with each other without conflict.

Although the spirit and principles of the present application have been described with reference to several embodiments, it shall be understood that the present application is not limited to the embodiments as disclosed, nor does the division of the aspects imply that the features in those aspects cannot be combined. The present application is intended to cover various modifications and equivalent arrangements included within the scope of the appended claims.

What is claimed is:

1. An implanted semi-implantable electrocorticography (ECOG) electrode comprising an electrode contact region and a welding pad region;
wherein the electrode contact region is provided on a first side of the electrode, and the electrode contact region comprises a plurality of electrode contacts;
the welding pad region is disposed on a second side of the electrode remote from the first side, and the welding pad region comprises a plurality of welding spots;
each of the plurality of electrode contacts and a corresponding one of the plurality of welding spots are connected by a conductive wire;
the electrode contact region comprises one or more hollowed-out portions each of which is disposed a position, except for an end portion of the conductive wire, between adjacent two of the plurality of electrode contacts, and the hollowed-out portion penetrates through a top surface and a bottom surface of the electrode contact region.

2. The implantable ECOG electrode according to claim 1, wherein the one or more hollowed-out portions comprise a plurality of hollowed-out portions, and the plurality of hollowed-out portions are arranged at intervals.

3. The implantable ECoG electrode according to claim 1, wherein the plurality of electrode contacts comprise small contacts and/or large contacts.

4. The implantable ECOG electrode according to claim 3, wherein the plurality of electrode contacts are the small contacts, the small contacts are arranged in an array, and every adjacent two of the small contacts are arranged at equal intervals;
wherein the plurality of electrode contacts are the large contacts, the large contacts are arranged in an array, and every adjacent two of the large contacts are arranged at equal intervals; or
wherein the plurality of electrode contacts are the small contacts and the large contacts, the small contacts and are arranged in an array and the large contacts are arranged in an array, the array of the small contacts and the array of the large contacts are arranged in a staggered arrangement, and every adjacent two of the small contacts are arranged at equal intervals and/or every adjacent two of the large contacts are arranged at equal intervals.

5. The implanted ECoG electrode according to claim 3, wherein an outer diameter of each of the small contacts ranges from 5 μm to 500 μm and an outer diameter of each of the large contacts ranges from 500 μm to 2500 μm.

6. The implantable ECOG electrode according to claim 1, wherein a width of the electrode contact region and a width of the welding pad region are both greater than a width of a middle region of the conductive line, and the width of the electrode contact region is greater than the width of the welding pad region.

7. The implantable ECOG electrode according to claim 1, wherein the electrode is provided as a sheet film, and the electrode comprises a flexible substrate, a metal layer, and an encapsulation layer;
wherein the flexible substrate is provided at a bottom of the electrode;
the encapsulation layer is provided on a top of the electrode;
the metal layer comprises the electrode contacts and the welding spots, and the conductive wire; the conductive wire is disposed between the flexible substrate and the encapsulation layer; and the electrode contacts and the welding spots are exposed out of the encapsulation layer;
the hollowed-out portion penetrates through a top surface of the encapsulation layer and a bottom surface of the flexible substrate in the electrode contact region.

8. The implanted ECOG electrode according to claim 7, wherein the implanted ECoG electrode comprises at least one of:
the thickness of the flexible substrate ranges from 0.1 μm to 100 μm, and the thickness of the encapsulation layer ranges from 0.1 μm to 100 μm; or
wherein the metal layer has a thickness ranging from 10 nm to 1000 nm.

9. A manufacturing method of a flexible electrode, the method comprising:
providing an electrode flexible support layer on a surface of a silicon wafer substrate;
disposing a photoresist on the electrode flexible support layer, wherein the photoresist covers a part of a surface of the electrode flexible support layer;
forming a first metal layer on the electrode flexible support layer via deposition, wherein the first metal layer comprises a metal layer on the photoresist and an electrode structure metal layer on the electrode flexible support layer, and the electrode structure metal layer comprises electrode contacts, welding spots, and a conductive wire between each of the electrode contacts and a corresponding one of welding spots;
removing the photoresist;
providing an encapsulation layer on the electrode flexible support layer, and removing portions of the encapsulation layer above the electrode contacts and the welding spots; and
peeling the electrode flexible support layer from the silicon wafer substrate to obtain the flexible electrode.

10. The method according to claim 9, wherein the disposing a photoresist on the electrode flexible support layer comprises:
after the photoresist is photoetched and developed on the electrode flexible supporting layer, forming the photoresist with a preset pattern.

11. The method according to claim 10, wherein the photoresist is formed by a positive resist inversion process, and a section of an edge profile of the photoresist with the preset pattern after being developed is inverted trapezoidal.

12. The method according to claim 9, wherein after disposing a photoresist on the electrode flexible support layer, the method further comprises:
performing ionization treatment on an upper surface of the electrode flexible supporting layer covered by the photoresist.

13. The method according to claim 9, wherein the forming a first metal layer on the electrode flexible support layer via deposition comprises:
forming the first metal layer on the electrode flexible support layer via deposition by a thin film deposition process.

14. The method according to claim 9, wherein the removing the photoresist comprises:
stripping the photoresist by heating a water bath using acetone or N-methylpyrrolidone, wherein the metal layer on the photoresist is stripped along with the photoresist.

15. The method according to claim 9, wherein the removing portions of the encapsulation layer over the electrode contacts and the welding spots comprises:
depositing another photoresist on the encapsulation layer, wherein the another photoresist covers a part of a surface of the encapsulation layer;
forming a second metal layer on the encapsulation layer via deposition, wherein the second metal layer comprises a metal layer on the another photoresist and a metal layer on the encapsulation layer;
removing the another photoresist;
etching regions of the encapsulation layer which are not covered by the second metal layer, such that portions of the encapsulation layer above the electrode contacts and the welding spots are removed.

16. The method according to claim 15, wherein after etching regions of the encapsulation layer which are not covered by the second metal layer, further comprising:
removing the second metal layer.

17. The method according to claim 15, wherein the regions of the encapsulation layer which are not covered by the second metal layer comprises a region corresponding to the electrode structure metal layer and an electrode slotted region;
wherein etching regions of the encapsulation layer which are not covered by the second metal layer, such that portions of the encapsulation layer above the electrode contacts and the welding spots are removed comprises:
etching the regions of the encapsulation layer which are not covered by the second metal layer, such that the portions of the encapsulation layer above the electrode contacts and the welding spots, and the electrode flexible support layer and the encapsulation layer corresponding to the electrode slotted region are removed.

18. The method according to claim 15, wherein etching regions of the encapsulation layer which are not covered by the second metal layer comprises:
etching the regions of the encapsulation layer which are not covered by the second metal layer by using a reactive ion etching process.

19. The method according to claim 15, wherein after forming another photoresist on the encapsulation layer, the method further comprises:
performing ionization treatment on an upper surface of the encapsulation layer covered by the another photoresist.

20. The method according to claim 9, wherein the method further comprises:
depositing a metal on the flexible electrode by electroplating, such that the electrode contacts are flush with the encapsulation layer or the electrode contacts are higher than the encapsulation layer.

21. The method according to claim 20, wherein each of the electrode contacts has a diameter between 10 μm and 1500 μm.

22. The method according to claim 9, wherein the method comprises at least one of:
a thickness of the first metal layer is between 1 nm and 2000 nm; or
wherein the electrode flexible support layer has a thickness between 1 μm and 1000 μm.

23. A computer-readable storage medium having programs stored thereon that, when executed by a single-core processer or a multi-core processor, causes the single-core processor or the multi-core processor to perform the method according to claim 9.

24. A flexible electrode manufactured by the method according to claim 9.
